(19) United States
(12) Patent Application Publication
(10) Pub. No.: US 2023/0404709 A1
(43) Pub. Date: Dec. 21, 2023

(54) SYSTEM AND METHOD FOR ORTHODONTIC TREATMENT TIMES WHILE MINIMIZING IN-OFFICE VISITS FOR ORTHODONTICS

(71) Applicant: Alta Smiles LLC, Fort Washington, PA (US)

(72) Inventor: Gary D. GIEGERICH, Fort Washington, PA (US)

(21) Appl. No.: 18/035,046

(22) PCT Filed: Nov. 2, 2021
(86) PCT No.: PCT/US21/57666

§ 371 Date: May 2, 2023

Related U.S. Application Data
(60) Provisional application No. 63/109,046, filed on Nov. 3, 2020.

(51) Int. Cl. A61C 7/00; A61B 1/247; A61C 7/14; A61C 7/20; G16H 20/00; G16H 80/00; G16H 40/40; G16H 10/60

(52) U.S. Cl. CPC A61C 7/002; A61B 1/247; A61C 7/146; A61C 7/20; G16H 20/00; G16H 80/00; G16H 40/40; G16H 10/60

(57) ABSTRACT

The preferred invention enables a hybrid approach to orthodontic care whereby the treatment for bracket and wire orthodontics can be performed in a combination of settings—sometimes in-office and sometimes at home. The initial phase of treatment involves evaluation and planning up to the point when a patient has braces placed on their teeth in a clinical setting and the initial wires are engaged with or mounted to the patient's teeth. From that point forward the patent's treatment preferably involves a combination of in-office visits and adjustments coupled with remote adjustments performed by the patient themselves, under the remote supervision of a clinician. When treatment is complete, the patient preferably returns to a clinical office and has the braces removed. The preferred invention improves the quality of care, the efficiency of the clinician, minimizes errors, speeds treatment times, expands access to care and is much more convenient for patients.

### CROSS-REFERENCE TO RELATED APPLICATIONS

The present application claims the benefit of U.S. Provisional Patent Application No. 63/109,046, filed Nov. 3, 2020 and titled, “System and Method for Orthodontic Treatment Times While Minimizing In-Office Visits for Orthodontics,” the entire contents of which are incorporated herein by reference in their entirety.

### BACKGROUND OF THE INVENTION

Orthodontic treatment utilizing bracket and wire techniques is a process of aligning teeth and fixing malocclusions. A typical treatment involves one or more of the following steps: taking diagnostic records, creating a diagnosis and treatment plan, determining whether any teeth need to be extracted and at what phase of treatment, determining bracket placement, affixing brackets, inserting wires, periodic wire changes/adjustments, use of inter-arch and intra-arch elastics to correct malocclusions, create spaces between teeth using spacers, close spaces between teeth using elastics, rotate teeth, upright teeth and level teeth, removing the brackets and wires, followed by a retention protocol using retainers to maintain the final tooth position. Each of the described recording, diagnosis, treatment, extraction, affixing, inserting, adjustment, evaluation, correction, removal, retention and related steps typically involve the patient's physical presence at an appointment with an orthodontist, dentist, technician or other medical professional. From the patient's perspective, these in-person appointments require interruption to work and educational schedules, creating a burden on the patient's and their caregiver's time and schedules, at times for check-ups that result in little to no modification or adjustments to the patient's treatment plan and hardware. This interruption can be particularly burdensome for patients who live in rural communities far from treatment locations or in any location where treatment locations are a significant travel distance from the patient's home location.

Typical treatment times for bracket and wire treatment are twelve to thirty (12-30) months. Treatment times can be shorter or longer depending on a variety of factors including the severity of the case, the treatment modality used, the skills of the clinician, intervals between adjustment appointments, patient age, patient compliance in following the treatment protocols, patient physiology, and the oral health and hygiene of the patient.

Treatment typically involves many in-person appointments to periodically adjust the braces, wires and elastics, etc. Appointments are scheduled in advance based on an estimate by the clinician as to when a modification to treatment (an adjustment) may be appropriate, as opposed to when the patient is literally ready for their next adjustment. Such assessment is traditionally done with an in-person visit with appointments spaced between four and twelve (4-12) weeks, depending on a variety of factors related to the patient and the clinician.

Large portions of the world lack access to orthodontic treatment. For example, in the United States with approximately ten thousand (10,000) orthodontic specialists, generally referred herein as “Orthodontists”, approximately fifty percent (50%) of the counties in the United States do not have any full-time orthodontist. The access is far worse in most other countries, such that patients who are not located near an Orthodontist, the patient either must travel long distances and spend significant amounts of time travelling to appointments over numerous years or must forgo orthodontic treatment. If they have access to orthodontic care at all, many people must travel substantial distances if they want treatment. Costs are high and access is very limited for many patients who need treatment.

Many of the appointments are not timed well and result in a determination by the clinician that nothing new needs to be done at that particular appointment, which is frustrating for patients who have spent significant time and effort to meet their appointment requirements. Consequently, patients spend large chunks of time getting to and attending appointments only to find that they didn't need to be there anyway.

Other appointments require refinements to the treatment that, prior to this invention, had to be done with in-person appointments with a clinician.

A typical orthodontic treatment, in accordance with best practices advocated by major industry associations follows the sequences of: (1) Initial appointment: (a) Initial diagnostic imaging, which may include radiographs, intra-oral images, external images of patient's face, (b) diagnosis and treatment plan created by clinician; (2) Bonding Appointment: (a) brackets are placed on the patient's teeth by clinician; (3) Adjustment Appointments: (a) a check-in phase where the patient is seen by the clinician before treatment is prescribed (Rarely does the adjustment appointment involve taking progress images of the patient's arches as the orthodontic hardware moves and shifts the patient's teeth toward a final position, (b) the clinician prescribes a treatment adjustment for that appointment, (c) the adjustment is completed by either the clinician or more commonly by a dental assistant), and (d) check-out phase occurs when the prescribed treatment is executed by someone other than the clinician, most commonly the dental assistant (The check-out occurs after completion of the treatment. The clinician again looks in the patient's mouth to ensure that the prescribed treatment is done correctly. Before sending the patient home); (4) Final diagnostic imaging parallels that of the initial consultations; (5) Debonding (The appliances are removed from the patient's mouth and the patient moves on the retention phase of treatment); (6) Retention wherein the patient is provided one of; a removable appliances or wires affixed directly to the lingual side of the teeth to keep the teeth from shifting post-treatment; and (7) Periodic check-in appointments to ensure that the teeth have not shifted.

It is desirable to design and implement an orthodontic treatment plan and schedule that reduces the amount of time and effort a patient is required to travel to and attend appointments that are often unnecessary and to generally improve treatment for orthodontic patients by utilizing novel treatment systems and methods. The preferred present invention improves orthodontic treatment systems and methods, addressing shortcomings of existing standard treatment systems and protocols.

### BRIEF SUMMARY OF THE INVENTION

The preferred invention is directed to a system and method for orthodontic treatment to accelerate treatment times while minimizing the need for in-office visits for bracket and wire orthodontics.

The preferred invention relates to an image capture method used during patient adjustment appointments for check-in and check-out. The preferred method relates to check-in and evaluation methods and processes, including the data gathered, communication of the results of the evaluation and the recommendation to the patient, and the means for the patient to make certain adjustments themselves, such as by providing custom length wires (either customized and delivered to patient or patient customizes uses our inventive techniques), unique wire stops, custom selected or custom made elastics tailored to the specific patient's needs. The elastics are not limited to custom elastics and may be selected from off-the-shelf elastics that are best suited for the particular treatment plan. The preferred invention also relates to the patient check-out process, where data is analyzed, and the results of the analysis are communicated to the patient efficiently and precisely. These techniques can also be used for in-office visits to improve the speed and accuracy of treatment.

Briefly stated, the preferred invention is directed to image capture and upload methods to gather initial information regarding the patient's initial clinical situation, patient check-in, evaluation of the data gathered during image capture and upload and check-in, communication of the results of the evaluation to the patient, distribution of customized wires and wire stops to the patient for installation and application of the wire's and stops to the patient's teeth and arches, preferably by the patient, distribution of customized elastics to the patient for installation and application of the custom elastics, preferably by the patient and check out. The elastics are not limited to being custom elastics and may be comprised of standard or off-the-shelf elastics that are selected based on a best fit and sizing for a particular treatment plan or adaptability to the preferred brackets and wire. The preferred check-in step can result in messaging from the central processor back to the patient for various reasons and indications, such as: (a) the patient needs to schedule an appointment, (b) a time or range of times when the patient may preferably schedule an appointment, (c) the patient has the option to schedule an appointment in-person or to make adjustments to the braces themselves, (d) customized recommendations regarding hygiene for the patient's teeth, gums and arches and (e) additional messaging regarding the patient or the treatment schedule.

In another aspect, the preferred invention is directed to a method for evaluating a patient's braces, including brackets and wires, teeth and gums at the conclusion of an orthodontic appointment. The method includes the steps of receiving images of the patient's arches, the images including left, center and right buccal images, evaluating the images at a central server, the evaluating involves comparing at least one of a predetermined number of brackets with a number of brackets identified in the images, a predetermined wire type and a predetermined wire length with a wire type and a wire length identified in the images, respectively, an elastic configuration on the brackets with a prescribed elastic configuration, a connection status of each of the brackets relative to the wire and a positioning of a patient's gum line relative to each of the teeth and transmitting a check-out message indicating one of whether the brackets are present, the wire length is approved, the brackets are connected to the wire, the elastic configuration is the prescribed elastic configuration and a patient gum status.

In a further aspect, the preferred invention is directed to a method for evaluating a patient's orthodontic treatment following application of a plurality of brackets to the patient's teeth and mounting a wire to the plurality of brackets wherein the plurality of brackets and wire are applied to the patient's teeth based on a first treatment plan. The method includes the steps of receiving images of the patient's teeth at a central processor from an image capture device, comparing the images of the patient's teeth with the plurality of brackets mounted thereon and the wire to the first treatment plan and previous images of the patient's teeth stored in the central processor, and transmitting a message from the central processor to the patient regarding an error condition or a confirmation condition. The images are captured with the assistance of an image capture guide that is configured to orient the image capture device relative to the patient's teeth and consistently space the image capture device relative to the patient's teeth. An updated treatment plan is developed based on the images of the patient's teeth.

In another aspect, the preferred invention is directed to a method for automated check-out of a patient performed after execution of an orthodontic treatment on the patient including application of brackets and a wire to the patient's teeth. The method includes the steps of receiving images of the patient's arches at a central processor and evaluating the images of the patient's arches at the central processor. The images include left, center, and right buccal images. The images are captured with an image capture device. The evaluation involves comparing the images of the patient's arches to a treatment plan stored in the central processor. The treatment plan includes a predetermined number of brackets, a predetermined bracket position and a predetermined archwire. The comparison includes identifying a number of brackets in the images of the patient's arches, a bracket position in the images of the patient's arches and an archwire in the images of the patient's arches. The comparison also includes comparing the predetermined number of brackets to the number of brackets, the predetermined bracket position to the bracket position and the predetermined archwire to the archwire. The central processor transmits a check-out message from the central processor indicating whether the predetermined number of brackets is equal to the number of brackets, the predetermined bracket position complies with the bracket position and the predetermined archwire complies with the archwire

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a flow chart of a system and method for orthodontic treatment times while minimizing in-office visits or appointments for orthodontic treatments in accordance with a preferred embodiment of the present invention;

FIG. 2 is a front perspective view of a 3D image or model of a patient's arches that may be produced based on the preferred systems and methods of FIG. 1, wherein brackets are mounted to the model teeth;

FIG. 3 is a bottom plan view of the 3D image or model of FIG. 2;

FIG. 4 is a top plan view of a preferred jig in accordance with a preferred embodiment of the present invention;

FIG. 5 is a side perspective view of an alternative preferred jig in accordance with an alternative preferred embodiment of the present invention;

FIG. 6 is a side perspective view of a stop and wire that may be utilized with the preferred system and method of the present invention;

FIG. 7 is a top plan view of a preferred cheek retractor that may be utilized with the preferred system and method of the present invention;

FIG. 8 is a top plan view of the preferred cheek retractor of FIG. 7, wherein an alternative arm is illustrated;

FIG. 9 is a side perspective view of an image capture guide of the preferred embodiment of the present invention; and

FIG. 9A is comprised of a series of views of an image capture device of the preferred system and method of the present invention.

### DETAILED DESCRIPTION OF THE INVENTION

Certain terminology is used in the following description for convenience only and is not limiting. Unless specifically set forth herein, the terms “a”, “an” and “the” are not limited to one element but instead should be read as meaning “at least one”. The words “right,” “left,” “lower,” and “upper” designate directions in the drawings to which reference is made. The words “inwardly” or “distally” and “outwardly” or “proximally” refer to directions toward and away from, respectively, the geometric center or orientation of the device and instruments and related parts thereof. The terminology includes the above-listed words, derivatives thereof and words of similar import.

It should also be understood that the terms “about,” “approximately,” “generally,” “substantially” and like terms, used herein when referring to a dimension or characteristic of a component of the invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

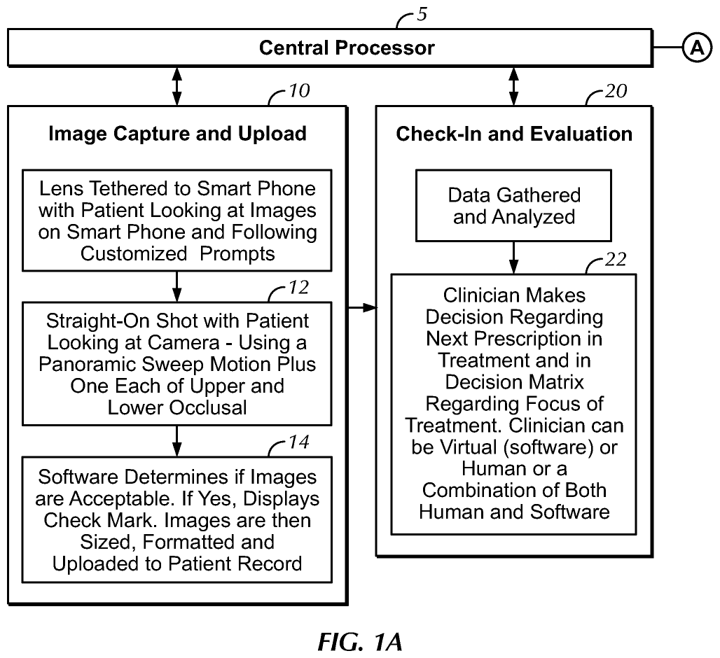

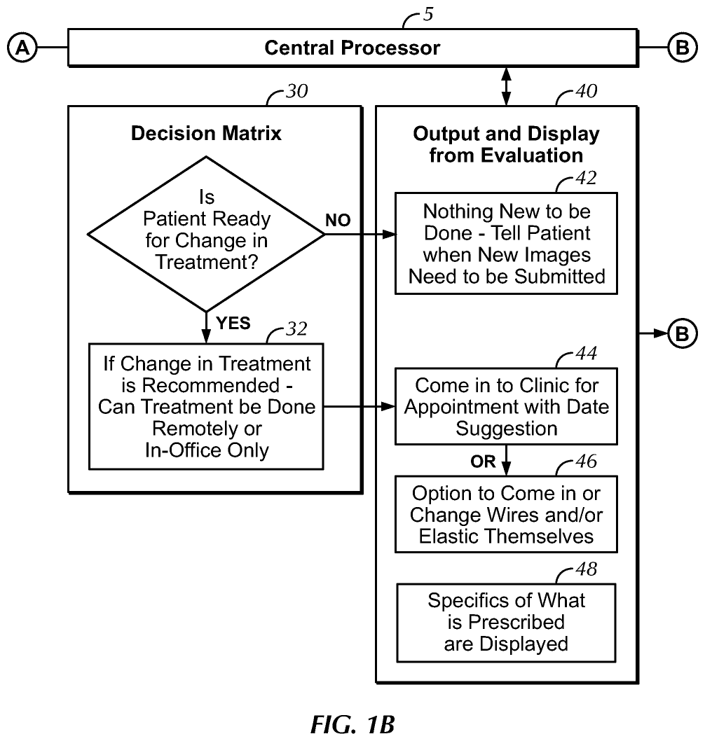

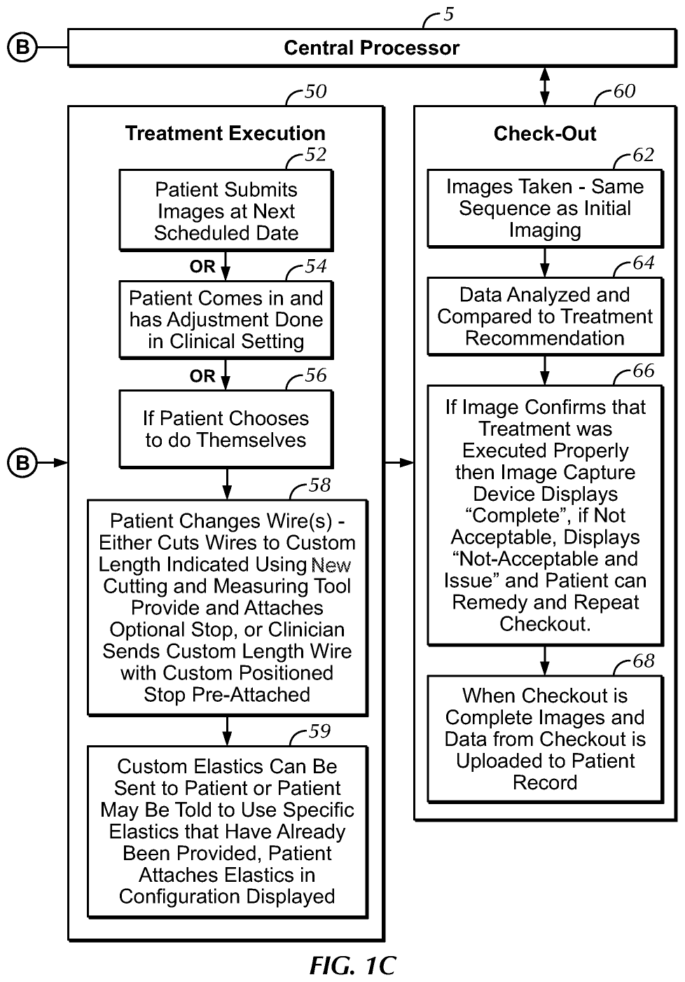

Referring to FIGS. 1-9, the preferred system and method may be separated into phases or steps of orthodontic treatment, which includes at least the initial diagnosis and treatment planning and placing of the braces on the patient's teeth. In this first preferred step images of the patient's teeth, arches and face are captured in an image capture & upload step 10. The preferred system and method also include taking diagnostic records and treatment planning of orthodontic treatment. The treatment planning may result from all imaging, diagnosis and treatment planning being done in-person up to the point of the patient receiving or having their braces 136, 138 applied to or bonded to their teeth and/or arches. The preferred system and method may also include gathering, storing and evaluating diagnostic records, including images of the patient's arches, which typically include intra-oral pictures—left, center and right buccal, mandibular occlusal and maxillary occlusal along with one or more of: a panoramic radiograph, cephalometric radiograph or 3-dimension cone beam computed tomography (“CBCT”) images. Patients may be able to do some of the initial, non-radiographic imaging from their home utilizing systems and methods of the preferred invention. The images, radiographs and other patient records are stored in a remotely accessible electronic file on a server or central processor 5 for relatively easy access and secure sharing with clinicians and the patient. The clinician utilizes software or software modules of the central processor 5 to record visually, and with precise measurements, the desired placement of each bracket 138 on each individual tooth and with this data stored in the patient record, as is described in greater detail below.

Once images of the patients arches, teeth, mouth and/or face are taken and transmitted to the central processor 5, a software module in the central processor 5 preferably creates a 3D image or model of the patient's arches 200 and allows the clinician to develop a treatment plan by placing or virtually placing the brackets 138 in their ideal location on the arches to effectively move the teeth during treatment. The modeled placement of the brackets 138 on the patient's teeth may be used to create indirect bonding transfer trays that are preferably ordered by the central server for shipment to the clinician or the patient for mounting to the teeth. The preferred indirect bonding transfer trays are custom fabricated to fit the patient's arch(es) and teeth so that all of the bracket positions are optimized based on the model and the computer simulation of treatment progression along with input from the treating clinician. The preferred indirect bonding transfer trays have recessed areas corresponding to each tooth into which the brackets are placed. The indirect bonding transfer trays are inserted into the patient's mouth and the brackets 138 are bonded to the patient's teeth. Once the bonding is complete, the indirect bonding transfer trays are removed, and the brackets 138 remain bonded to the teeth. The 3D image or model of the patient's teeth or arches 200 and the brackets 138 can also be used as a visual guide for on-site clinicians to use a reference for placing and bonding the brackets 138 by hand either during initial bonding or to re-bond or reposition a bracket 138 at any time during treatment.

In-person appointments may involve pre-imaging, treatment plan determined by central processor 5, delivery of brackets 138, wires 136, stops 140, elastics to clinician, meeting receptionist with package including everything for appointment, taking package to assistant to apply hardware to the patient's teeth, imaging for check-out purposes & done. Quick and more efficient that imaging and evaluation while patient waits at office.

In an image capture and upload step 10, the patient preferably takes images of their teeth, arches and face with an image capture guide 110, such as a camera 118, smartphone 118, tablet, x-ray machine or another image capture guide 110. The camera 118, smartphone 118 or tablet may be tethered to a lens, hardware, cheek retractor, track or other system that is able to consistently position the camera 118, smartphone 118 or tablet for collecting images. The patient may collect the images while observing the images being taken on a screen on the camera 118, smartphone 118 or tablet and following customized prompts on the screen that are received from the central processor 5. The patient preferably takes a straight-on shot with the patient looking at the camera 118, using a panoramic sweep motion or an inverted panoramic sweep motion with the camera 118 traversing a generally U-shape around the patients teeth, arches or face, plus one each of upper and lower occlusal images 12. The upper and lower occlusal images 12 may be captured via assistance from a mirror 22, which is described in greater detail below or by a custom image capture guide or device 110, 118 or technique. The inverted panoramic sweep is preferably made in a generally U-shape around the patient's face, arches and teeth to capture a panoramic image and the sweep is inverted, as the image is take facing the inside of the U-shaped sweep, as opposed to a conventional panoramic image where the image is taken with the camera facing outwardly of the typical arcuate sweep. This inverted panoramic sweep may be guided by a mechanism attached to the camera 118 or the patient to guide the image in the inverted sweep to consistently capture the teeth, face and arches of the patient at a consistent distance, angle and sweep length, but the use of the mechanism is not limiting and the patient or an assistant may perform the inverted U-shaped sweep without aid of the guiding mechanism. The images are preferably uploaded to the central processor 5 or a software routine stored in the image capture device 118, which may be comprised of a camera 118, smartphone 118, tablet or other image capture device determines if the images are acceptable and if the images are acceptable, a check mark is displayed, the images are sized, formatted and uploaded 14 to the patient record through or in the central processor 5.

In a preferred check-in and evaluation step 20, the data, including the gathered images are analyzed by the central processor 5 or the local software. The clinician, which may include the local software or the central processor 5, makes a decision regarding next prescription in treatment and in a decision matrix regarding locus of treatment, wherein the clinician can be virtual (software) or human or a combination of both human and software 22. In a decision matrix step 30, a determination is made whether the patient is ready for a change in treatment. If the patient is ready for the change in treatment or the change in treatment is recommended based on the image capture and upload step 10 or the check-in and evaluation step 20, a determination is made regarding whether the treatment can be conducted remotely or in-office 32. In an output and display from evaluation step 40, an indication is displayed to the patient that nothing new is to be done and the patient is told when new images need to be submitted 42 if the decision matrix determines the patient is not ready for a change in treatment. If the decision matrix step 30 determines a change in treatment is recommended, the patient is notified by the central processor 5 or the local software to come into the clinic for an appointment with date suggestion 44, the patient is given the option to come in for a change in treatment or to change wires and/or elastics themselves 46. The date suggestion preferably includes a recommended date for an upcoming appointment, a series of possible recommended dates that the patient may select for a subsequent appointment or a recommended range or days or times when the patient may schedule an appointment for a subsequent treatment. The output and display from the evaluation step 40 also preferably involve display or messaging to the patient regarding specifics of what is prescribed 48.

In a preferred treatment execution step 50, the patient may submit images at the next scheduled date 52, the patient may come into the clinician for an appointment and has an adjustment completed in a clinical setting 54 or the patient may elect to conduct the adjustment themselves without requiring a clinical appointment 56. If the patient elects to conduct the adjustment themselves, the patient preferably changes wires 136, including cutting wires 136 to custom length 58 indicated using a cutting and measuring tool 150, 150′, attaches optional stops 140 or the clinician sends custom length wires 136 with custom pre-attached stops 140. The patient may also apply custom elastics that are sent to the patient by the central processor 5 and may be told to use specific elastics that have already been provided such that the patient is able to attach the elastics in the configuration displayed on a monitor by the central server 5 or the local software to the patient 59. The central server 5 may also select standard or off-the-shelf elastics for the determined treatment plan for delivery to the patient so that the patient is able to apply the elastics to the braces based on the identified configuration or treatment plan.

In a check-out step 60, images are taken 62, preferably the same or similar images as were taken in the image capture and upload step 10. The images and any additional data from the patient's records are compared to the treatment recommendation 64. If the images and additional data analyzed by the local software or the central processor 5 confirms that the treatment was executed successfully, then the image capture device receives a message from the local software or the central processor 5 that the check-out step 60 is “complete” and if the local software or the central processor 5 determines that the treatment was not executed successfully, a “not-acceptable and issue” message is displayed to the patient and the patient is provided with instructions regarding how to remedy and repeat the checkout 66. When checkout is complete, images and data from the checkout is uploaded to the patient record 68.

In the preferred system and method, the patient has braces affixed to their teeth or arches and the initial wires 136 may be engaged into the brackets 138 by a clinician. The patient then typically participates in periodic check-in and evaluation of the hardware, their teeth and their arches. Periodically (can be at times specifically mandated by the clinician or on a preset schedule), the patient or a friend or family member will take new images of the patient's arches without coming into the office and upload them to their patient record and to the central processor 5. The images preferably include one or more of: a panoramic view of all of the patient's teeth, left buccal, center, right buccal, maxillary occlusal and mandibular occlusal. The images are preferably processed through a proprietary software application or software modules of the central processor 5 that guides the user through an image capture routine and edits, enhances, records and stores the images in the patient's records at the central processor 5.

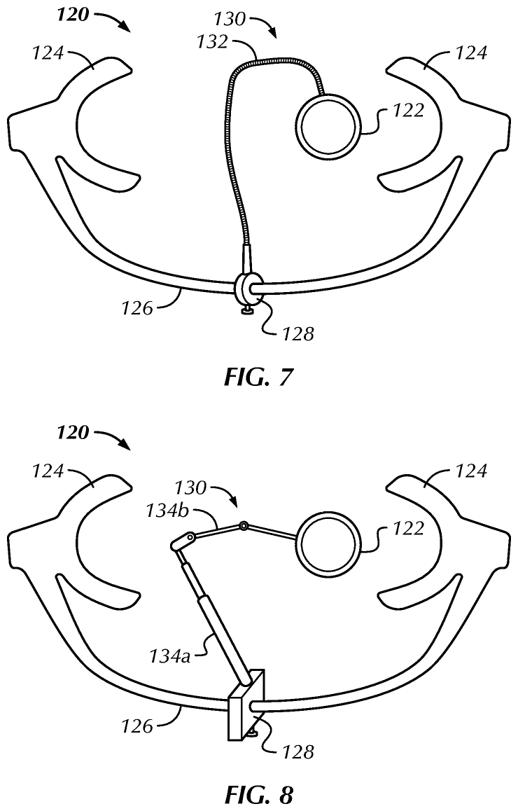

Referring to FIGS. 7 and 8, in a preferred method and system, the images may be captured utilizing a novel cheek retractor 120 employed or used by the patient to create distance between the teeth and gums to produce a relatively clear and consistent image or images for the patient's records and for the software application of the central processor 5. The patient preferably takes images of their arches themselves using an image capture device 118, which may be comprised of a camera connected to an electronic device such as a smart phone or tablet. The camera 118 may be tethered to the electronic image capture device by a cord, may be wirelessly tethered or connected via blue tooth or may use some other wireless means of communication to tether the camera 118 to the electronic image capture device or the central processor 5. The camera 118, smart phone 118, tablet or other image capture device 118 may be mounted to the electronic image capture device, which may be integrated with the cheek retractor 120, to consistently position the camera 118, smart phone 118, tablet or other image capture device 118 relative to the patient's teeth and arches to further produce consistent images for use by the software. The novel cheek retractor 120 may include a mirror 122 attached thereto to capture images of the lingual side of the teeth or other difficult to view portions of the teeth and may also include a light or flash that illuminates an area for clear image capture. The mirror 122 is particularly useful in capturing occlusal images of the patient's arches and teeth. The occlusal images are transmitted to the central processor 5 for analysis by the software modules and development of the treatment plan for the patient. The occlusal images may also be utilized for upcoming appointment scheduling and timing for scheduling the next appointment, such as by identifying eruption of the patient's teeth that may only be determined based on the occlusal images. The patient can look at a screen on the electronic devices 118 while taking the images and follow a software directed image capture protocol. The cheek retractor 120 may further include arms, tracks, hinges or other mechanical systems that guide the patient when capturing the images of their teeth and arches, particularly for images where a panoramic or full arch image is captured that is preferably captured by moving the cameras 118, smart phone 118, tablet or other image capture device 118 relative to the patient's teeth, arches and/or face. The cheek retractor 120 is not limited to including the described arms, tracks, hinges or other mechanical systems and the preferred system and method may be operated without such mechanical assistance.

The cheek retractor 120 of the preferred embodiment includes opposing cheek guards 124 and a central arm 126 connecting the opposing cheek guards 124. A slidable clamp 128 is attached to the central arm or retractor arm 126 with a spacing arm 130 extending to the mirror 122. The spacing arm 130 is preferably attached to the slidable clamp 128 at a proximal end and to the mirror 122 at a distal end. The spacing arm 130 may be comprised of a gooseneck arm 132 that may be manipulated by the user to position the mirror 122 relative to the clamp 128 and central arm 126 so that the mirror 122 may be positioned for capturing images of the patient's teeth. The spacing arm 130 may alternatively be comprised of a proximal telescopic arm 134a and a distal pivot arm 134b with the mirror 122 attached to a distal end of the distal pivot arm 134b. The combination of the proximal telescopic arm 134a and the distal pivot arm 134b facilitates positioning of the mirror 122 relative to the patient's teeth when the cheek retractor 120 is mounted to the patient. The slidable clamp 128 is preferably slidable along the central arm 126 to orient and position the mirror 122 relative to the cheek guards 124 and the central arm 126 for positioning and angling the mirror 122 relative to the patient's teeth. The preferred check retractor 120 may also include an illuminator (not shown), such as light-emitting diodes (“LED”) or other lights, to project light onto the patient's teeth, arches and face such that images are not shaded or dark. The illuminator may shine at different sequences or patterns that track the movement and position of the image capture device or camera 18 so that a particular area of the patient's teeth, arches and face are illuminated as the image capture device or camera 18 collects images.

Referring to FIGS. 1-9, the patient preferably takes multiple images of their teeth, arches and/or face, including at least one of 1) a panoramic picture that is spliced by the software into the three images (right, left and center) and/or 2) upper or maxillary occlusal and/or lower or mandibular occlusal images. The software on the electronic device preferably ensures that all images are captured using the image capture protocols and that the images are properly sized, properly oriented, and focused for maximum clarity and the central processor 5 preferably quality checks the images to ensure appropriate images and quality are obtained during the image capture and upload step 10. Once the software module and/or the central server 5 has determined that the images meet a minimum acceptable standard, the patient is preferably notified on screen at the camera 118, smart phone 118, tablet or other patient interface and the images are preferably automatically uploaded to the patient record at the central server 5.

The preferred embodiments of the described system and method utilizes hardware and form factors of the image capture routine to develop the model for bracket mounting and a treatment plan for the patient. The preferred embodiment involves use of a high-definition camera or image capture device with resolution in the range of 1920×1080 or 1280×720 with primary focusing distance of one to four inches (1-4″) but is not so limited and may have alternative resolutions and ranges, as well as preferred image focusing distances. The preferred imaging device has a seventy-degree (70°) front lens and a seventy-degree (70°) side lens but is not so limited and may have alternative imaging devices having a different field of view. A plurality of adjustable light-emitting diode (“LED”) lights or other illuminating systems or methods (not shown) are mounted in a pattern, preferably a circular pattern, around the lens or camera 118 to provide consistent and adequate lighting for capturing images of the patient's teeth, arches and face. In the preferred embodiment no physical device is required to steady the patient's head while taking the images nor is there a mechanical device connecting the cheek retractors to the lens or camera 118, but the system is not so limited, as is described in further detail herein.

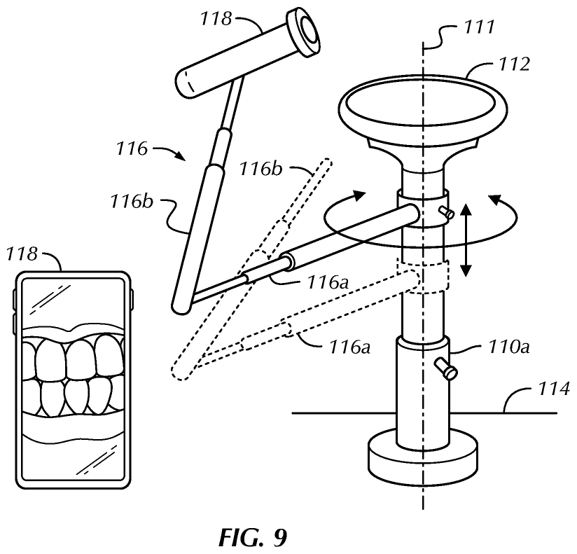

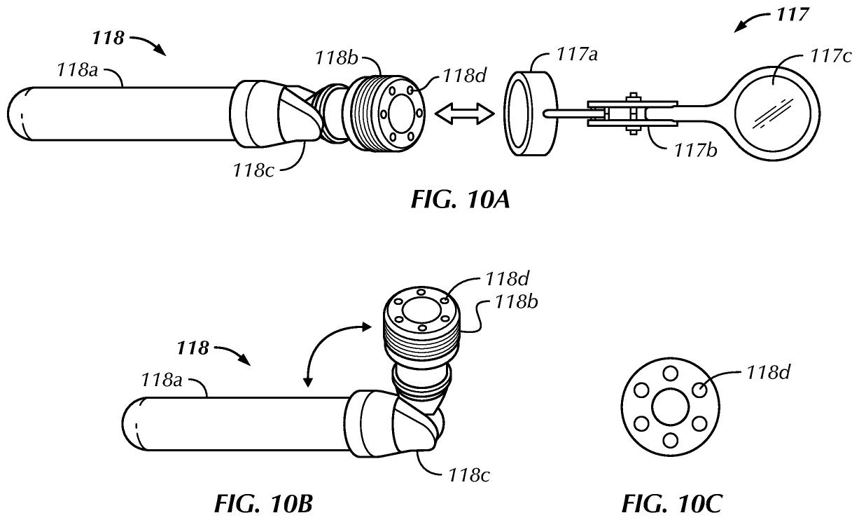

Referring to FIG. 9, the preferred invention also contemplates an alternative embodiment which incorporates a mechanical image capture guide 110 that is incorporated into the preferred image capture technique. The image capture guide 110, which can be placed on a flat surface 114, includes a fixed chin rest 112 on which the patient can comfortably place their chin and may also include an actuating arm 116 connected to the chin rest 112 on which the camera 118 or image capture device 118 is connected. The actuating arm 116 preferably guides the image capture device 118 to slide or move horizontally along a plane of the patient's arches to preferably capture center and buccal images and may also move vertically from a centered position to capture occlusal images. The preferred image capture guide 110 provides enhanced stability during image capture. The image capture guide 110 preferably includes a pedestal 10a that rests on the flat surface 114 for stability and the pedestal 110a may telescope vertically relative to the chin rest 112 to facilitate positioning of the chin rest 112 relative to the flat surface 114 for differently sized patients and positions of the flat surface 114. The actuating arm 16 of the preferred embodiment includes a horizontal arm 16a connected to the pedestal 110a or the chin rest 112 and a pivoting arm 116b connected to a distal end of the horizontal arm 116a. The horizontal arm 116a preferably extends generally horizontally from the pedestal 110a or the chin rest 112, generally parallel with the flat surface 114, and may include telescoping features to adjust the horizontal position of the distal end of the horizontal arm 16a from the chin rest 112. The pivoting arm 116 is preferably pivotably connected to the distal end of the horizontal arm 116a, is pivotable relative to the horizontal arm 116a and may have telescoping capabilities. The horizontal arm 116a may be connected to the pedestal 110a or the chin rest 112 to pivot about a vertical axis 111 of the image capture guide 110 such that the camera or image capture device 118 is guided along an image capture path to collect desired images of the patient's arches, teeth and/or face. The image capture device 118 may be comprised of a custom camera, smart phone, tablet or any device 118 that is able to collect and transmit images, perform the preferred functions described herein, withstand the normal operating conditions of the image capture device 118 and generally operate in accordance with the described, preferred system and method.

Referring to FIG. 9A, the preferred system and method may also utilize an alternative preferred image capture device 118 having a shaft 118a, a head 118b, a pivot joint 118c connecting the shaft 118a to the head 118b and an illumination feature 118d on the head 118b. A mirror mechanism 117 is removably mountable to the head 118b and preferably includes a connector 117a, a pivot arm 117b and a mirror 117c on a distal end of the pivot arm 117b. The alternative preferred image capture device 118 may be utilized with the image capture guide 110 or may be utilized independently to collect images of the patient's teeth, arches and face for transmission to the central processor 5. The pivot joint 118c permits pivoting of the head 118b at preferred angles to capture images of the patient's teeth and arches, particularly to capture difficult to view portions of the patient's teeth that are obstructed by soft tissue, teeth or other anatomy, such as the lingual side of the teeth, mandibular occlusal images and maxillary occlusal images. The illumination feature 118d provides illumination for the target area of the images. The illumination feature 118d of the alternative preferred embodiment is comprised of six LEDs positioned around the perimeter of a front face of the head 118b, although the illumination feature 118d is not limited to being comprised of six LEDs and may be comprised of nearly any feature that is able to illuminate the target area for taking images, withstands the normal operating conditions and performs the preferred functions of illumination feature 118d of the alternative preferred embodiment. The mirror mechanism 117 is preferably removably mounted to the head 118b by a threaded connection but is not so limited and may be otherwise removably secured to the head 118b, such as by fastening, bonding, clamping or otherwise securing to the head 118b and may be integrally formed or permanently connected to the head 118b. The mirror 117c is preferably pivotable relative to the connector 117a and the head 118b in a mounted configuration to position the mirror 117c for reflecting the desired image from the mirror surface into the head 118b to capture the desired image, such as lingual images of the patient's teeth. The mirror 117c may be utilized to particularly capture lingual images, mandibular occlusal images and maxillary occlusal images. The orientation of the mirror 117c may be adjusted by the pivot arm 117b and/or by rotating the mirror 118b relative to the pivot arm 117b to reflect the desired imaged to the head 118b. The image capture device 118 and mirror mechanism 117 are not limited for use in the preferred invention and may be otherwise designed and configured to collect the desired images of the preferred system and method. The central processor 5 may send instructions to the user regarding how to utilize the alternative preferred image capture device 118 and the mirror mechanism 117 to capture desired images of the patient's teeth, arches and face.

Referring to FIGS. 1-9, the preferred image capture technique for capturing images of the patient's teeth, arches and face is relatively easy for the patient or an assistant, can be employed nearly anywhere without requiring the patient to physically attend an in-person appointment or visit with a clinician, can be completed by the patient themselves, thereby improving convenience for the patient and reducing unnecessary time burdens for clinician, ensures that each desired image is captured by the patient, and preferably ensures consistent, high quality of the images.

In the preferred embodiment, the patient's orthodontic needs, requirements and potential treatment plans are evaluated once the images are uploaded to the patient record on the remote server. The images are preferably evaluated by a computer program using at least one of: image processing, artificial intelligence (“AI”) and machine learning in the central processor 5. The images are evaluated for a number of factors to determine at least one or more of the following: (1) which wires 136 are in place and the sizes of the wires 136 (2) whether all brackets 138 that should be attached are still attached and whether their current position is consistent with the computer modeling stored in the patient record that identifies the preferred initial position (note that positions can be updated throughout treatment, if brackets 138 are “re-positioned at the direction of the clinician), (3) whether each bracket type 138 is properly engaged, (4) whether there is evidence of gum swelling or recession or other concerns related to the gums, which may include an assessment of the severity of the condition, (5) distances between each proximal tooth, including one of the following dimensions—the incisal edges, the first visible section of the tooth as it protrudes from the gum and the mid-point between the incisal edge and first visible portion of the tooth, (6) the visible size of each patient's teeth—height, width and surface area, (7) distance between the center-point of wire slot for each bracket 138 from the center-point of the slot of the proximate tooth, (8) overall length of the patient's arch and length of wire 136 that would be needed to be engaged into the brackets 138, and (9) if molar tubes are used, the extra length of wire required to first insert the wire 136 into the molar tubes then push the wire 136 into each bracket slot. After results of the evaluation are displayed in patient record, a size of patient arches, as measured in several ways including from the distal point of each molar to the midline of the arch, and/or distances between teeth information may be optionally displayed to the patient.

The preferred system is also able to determine what wires 136 are being inserted into the patient's mouth, the material of the wires 136 and the thickness or diameter of the wires 136. The wires 136 themselves are preferably of nitinol and/or stainless steel in gauges from eight thousandths to sixty-four thousandths of an inch (0.008-0.064″), more preferably twelve thousandths to twenty thousandths of an inch (0.012-0.020) in either round or rectangle shapes. When the wires 136 are constructed of nitinol, the wires 136 are preferably pre-formed in the shape of a patient's arch but are not so limited and may be shaped and formed by the clinician and/or patient. Based on the images collected by the preferred system, a software module of the central processor 5 may detect the diameter, shape, material, and related features of the wires 136 via imaging to determine their material and/or gauge and other features, such as: (a) a coating on a section of the wire 136 in a distinct color that will dissolve or fade relatively quickly so that it is not visible (unsightly to patient) and the preferred coating is benign to human ingestion and biocompatible, (b) a coating on the wire 136 in the non-visible light spectrum covering all or a portion of the wire 136, (c) a system of placing printed lines on the wire 136 similar to a bar code type method, wherein the lines correlate to specific wires 136 and the lines may be printed using visible inks or inks not visible to the human eye, such that the coating may include a bar-code like series of lines to denote the type of wire, (d) a permanent color marking positioned on only a small portion of the wire 136 so that it is not easily visible during typical use, (e) an indentation or other permanent marking on the wire 136 and other features to identify the wire 36, such as determining wire diameter based on the collected images and scaling the images. A software module of the central processor 5 may be programmed with the ability to measure the diameter and/or shape of the wire, indicators or colorings that facilitate identification of the material of the wires 136 based on color, sheen or texture, the ability to identify any coatings or printing on the wire and the ability to identify indentations or other physical markings on the wires 136.

The preferred system and method are also able to monitor and determine whether the wires 136 are properly engaged into the brackets 138 prior to and following an appointment with a clinician or at interim times between appointments. Treatment typically involves application of one or more of the bracket types listed below, each of which engages the wire in a unique way. The preferred invention involves using high resolution imaging and evaluation by the software modules of the central processor 5 to identify error conditions or determine (a) in the case of the self-ligating brackets 138 that a cross-section of the archwire 136 engaged into each bracket 138 is not visible which typically only happens if the “gate” of the bracket 138 is fully closed, thus obscuring that section from view, or (b) in the case of a traditional bracket 138, where the elastic ligature used to engage the bracket 138 is engaged securely. The system may also be configured to identify error conditions or confirm that the gate of the bracket 138 is closed, thereby confirming that the wire 136 is engaged with each of the brackets 138, as opposed to only identifying open or improperly configured gates at the conclusion of the appointment. Normally engagement would be from four points or “wings” around the bracket 138, but a minimum of two points of engagement may be sufficient. In the case of molar brackets 138 that the wire 136 is adequately engaged into molar bracket 138 but that the wire 136 does not extend distally beyond the bracket 138 so much that the wire 136 pokes the patient's checks and/or gums.

Absent the preferred invention, in order for a clinician to evaluate the proper positioning of the brackets 138 and the wires 136, in-person, the clinician normally is required to manually look at and “test” each bracket 138 using either their fingers or a handheld dental tool to ensure that the bracket 138 is properly engaged with the wire 136. This manual evaluation is time consuming, interrupts the flow of patient treatments in the office since normally there are multiple patients receiving treatment in the clinician's office at the same time and may be uncomfortable for the patient with the continued presence of fingers and instruments in the patient's mouth. This manual evaluation technique can lead to: (a) some clinicians skipping this important step or (b) errors due to the small sizes of the brackets 138, the positioning of the brackets 138 in the mouth, fatigue of the clinician and the large number of individual tooth checks necessary per patient and per day by the clinician. This failure of the manual quality check is particularly a concern when many patients are being treated in an office on a daily basis.

The preferred system and method permit measurement of a visible portion of an intersection and surrounding area between each tooth and the adjacent gum line and comparing these individual areas to the images and measurements from prior appointments to identify error conditions when gum swelling or disease is identified. If the gum and gum line is swollen, the gums will obscure more of the tooth and less tooth surface will be visible in subsequent images when compared to previous images. Conversely if the gums are recessed, the opposite effect will occur. The software module of the central processor 5 is also able to compare the color and hue of the gums to one or more of: (a) prior appointment images for the patient or (b) with a database of “normal hue” for a healthy gum (compiled from a wider patient population). Information from this evaluation can be displayed in the patient record, which is preferably stored in the central processor 5. Storage of the sequential evaluations enable the clinician and/or software module of the central processor 5 to visually show the patient the specific area of the gums which are of concern, if any, and recommend proactive measures to improve the condition identified. The preferred system may also notify the patient that the gums and intersections between the gums and teeth are normal, and no extra attention is required, nor treatment recommended.

The preferred system and method may also be employed in cases of mixed dentition, wherein the patient has some primary or “baby” teeth and some permanent teeth. In the mixed detention cases, the preferred system and method can measure how much a permanent tooth has erupted from the gums relative to prior imaging to assist with development of a treatment plan. This measurement of eruption can be helpful in determining when a bracket 138 can safely be bonded to a tooth and/or when a previously bonded bracket 138 may be repositioned on a larger surface area portion of the tooth. In young patients, teeth are still erupting/coming in and the clinician or patient are unable to bond the bracket 138 to the tooth until the tooth fully in or mostly extending into the patient's mouth. The preferred system and method utilize the collected images for multiple appointments or image collection sessions to track progressing of eruption/coming in of teeth and when to apply a bracket 138 to the new tooth. The sequential images collected by the preferred system and method may also indicate to the clinician or the software module that a particular tooth may be ripe for extraction, gum laceration to aid eruption of the tooth, bonding of the bracket 38 to the partially erupted tooth to expedite full eruption of the tooth or other treatments based on the sequence of images and the treatment plan.

The preferred wires 136 have a fixed or static length when installed in the patient's mouth and mounted to the brackets 138. When the teeth start moving based on the forces applied by the brackets 138 mounted to the wires 136, thereby straightening and moving closer together, the archwires 136 may move distally through the molar bracket 138. This movement of the wire 136 during treatment may result in the wire 36 poking or otherwise aggravating the patient's cheeks and gums and the ends of the wire 136 extend further out of the molar bracket 138. The preferred system and method facilitate notification to the patient or clinician of the error condition that the wires 136 may aggravate the patient after a predetermined time and may recommend an appointment for trimming the wire 136 or suggested trimming of the wire 136 by the patient.

This preferred evaluation method is much faster than conventional evaluation where the patient attends the in-person appointment or visit, more accurate in terms of quality control, and data regarding the size of teeth and the distances between the teeth are consistently collected and reviewed for changes by the central processor 5. The preferred evaluation does not require any hands-on work by the clinician, which at times get skipped and provides an oral health assessment of the patient's gums based on prior conditions and a broader population evaluation. The preferred evaluation can be done at any time, rather just during office hours, can be done remotely and provides information as to what custom sized wire is required and where a stop 140 should be on the wire 136 for the patient based on the collected images.

In the preferred embodiment, the software module of the central processor 5 or a consulting orthodontist recommends a treatment for the patient based on the captured images. The human or software (this would relate to the AI and machine learning techniques) determines and records: (a) whether a change in treatment is required, (b) when a change in treatment will be required, c) the treatment recommendation, and d) whether the treatment can be done only in a clinical setting or may be done by patient, without visiting a clinic. The treatment recommendation preferably displays in the patient's record and/or is communicated to patient via a message directly from the central processor 5 (if off-site). If the recommendation is that the treatment can be done at home or off-site by the patient, the off-site recommendation is electronically communicated to the patient by the central processor 5 and the patient then has the option as to whether they want to come to the clinic to receive treatment or conduct the immediate step in the treatment plan themselves at home or at their preferred off-site location.

The preferred treatment is executed based on the determinations made by the central processor 5 or preferences of the patient. Certain of the treatments are preferably performed in a clinical setting with the clinician, particularly if the treatment recommendation is that the patient come to the clinic to receive their next treatment. In this situation, the patient preferably goes to the clinic to get their treatment within a time recommended by the clinician. During the in-office, prior to the appointment, or in the clinical setting visit or appointment, the software or central processor 5 may display a customized wire size that makes it easier for the clinician to trim the archwires 136 more efficiently to expedite the patient's in-office or clinical appointment. During the in-office treatments patient images may be retaken and uploaded to the patient record in the central processor 5, or the clinician may determine that the checked-in images provided by the patient are recent enough and sufficient, such that new images are not required.

Portions, preferably a large majority, of the treatments may be performed at home or remote from the clinical setting by the patient to improve convenience and save significant time for the patient. If the treatment is to be done at home or remote, the treatment may involve changing archwires 136, intra-arch elastics or inter-arch elastics. The intra-arch or inter-arch elastics may be custom or standard elastics that are mapped for location so that the patient is directed to the desired positioning of the elastics for the treatment plan. To change a wire 136, a remote office is directed by the central processor 5 to provide a specific or customized wire 136 to the patient. The specific or customized wire 136 prescribed by the central processor 5 may be defined based on thickness, material, customized length and other features to fit the patient's mouth properly based on calculations determined during the evaluation procedure. Such calculations preferably consider the overall length of the arch as well as additional length of wire 136 required to engage each bracket 138. The patient may be provided archwires 136 incrementally throughout treatment or in some other manner. The archwires 136 are preferably, sufficiently precise that, once inserted, there is no need to cut the distal ends 136b as is done currently with orthodontic treatment. Patients may, however, still be taught or instructed by the central processor 5 how to trim the distal ends 136b using a cutting mechanism or cutting device 156, as is described in greater detail herein.

Details about alternative methods of the patient customizing the wire 136 themselves include systems and techniques for measuring the wires 136 and cutting the wires 136 themselves and comparing the size to what's displayed on the screen from the central processor 5 to the physical wire 136 received by the patient. In traditional orthodontics, the orthodontic professional or clinician “eye-balls” the wire 136 when trimming the wire 136 to its final desired length for fitting to the patient's teeth. In current treatment techniques, measuring and changing archwires 136 is done as a multiple-step, inefficient and often inaccurate process. An oversized wire 136 is typically used as a one-size fits all length and this oversized wire is cut gradually to size. The clinician conducts a visual assessment of the length needed for the patient and trims the wire 136 on one or more distal ends 136b. The clinician then preliminarily assesses if the trimmed wire 136 fits appropriately in the patient's mouth, usually by putting the wire 136 proximate to the arches and doing a visual assessment. If the preliminary assessment indicates that the wires 136 are still too long, the clinician cuts the wires 136 again and tries again, repeating this process until the appropriate size is realized. This process is often repeated multiple times. Once a visual assessment indicates that the wire length seems correct, the clinician places the wire 136 into the brackets 138. Once the wires 136 are placed and engaged into brackets 138, the wires 136 normally require a final “trimming” on the distal ends 136b to ensure that the wire 136 is not poking into the patient's checks, thereby causing significant discomfort, lacerations and potentially exposing the patient to infections. Due to the inexact nature of this approach, the patient is often poked by the wires 136 (with the associated great discomfort) while the wire 136 is being engaged into the bracket 138 prior to the final trimming.

The stop 140 is typically required for attachment to the wire 136 to keep the wire 136 from sliding on the brackets 138. When the treatment involves the use of self-ligating brackets 138, the wires 136 can slide within the brackets 138 (and potentially cause several problems) unless a feature is employed to keep the wires 136 from sliding relative to the brackets 138. In a clinical setting, the clinician typically applies a crimped piece of metal onto the archwire 136 between one or more of the brackets 138 to keep the wire 136 from sliding. A patient at home cannot reasonably successfully perform the wire crimping process, as the procedure involves putting very small pieces of metal onto a tiny wire 136 in a precise location using a handheld medical instrument. The preferred embodiment of the present invention involves an alternative system and method that is user-friendly and relatively simple to employ by the patient to prevent sliding of the wires 136.

In the preferred embodiment, pre-mounted stops 140 are installed on the archwires 136 by the clinician based on the measurements provided by the software modules of the central server 5 based on the images uploaded to the central server 5 by the patient. The stops 140 are custom-positioned on the archwire 136 for the patient's unique arch length so that the stop fits between one or more brackets 138 that are already mounted to the patient's teeth. The stops 140 may be at least one of: a) rigidly affixed, and b) affixed such that with some manual force they can slide on the archwire and then be crimped permanently with a crimping instrument, preferably after wire installation. The stop 140 is described in further detail below.

In the preferred embodiment, the patient can change elastics on their braces 136, 138. If an elastic change is prescribed by the clinician, which includes the software associated with the central processor 5, the clinician decides as to which configuration and size of elastic to use based on the imaging and the measurement data provided by the evaluation of the image from the patient's records and/or direct physical examination of the patient. The precise measurement data provided by the Evaluation enables the clinician to choose the appropriate length and tension elastic for the patient's particular treatment plan. These elastics may be custom prescribed or standard, off-the-shelf elastics for application to the patient's braces at locations determined by the central processor 5 and communicated to the patient via a treatment plan. The patient is preferably provided with instructions regarding: (1) which elastic is to be used, (2) which posts (anchor points extending outwardly (mesial)) commonly referred to as “posts,” which are to be used to attach the elastics, (3) instruction as to how to apply the elastics, (4) a tool with which to apply the elastics, (5) instructions regarding use/wear of the elastics and any additional information that may be provided to the patient regarding use, installation and maintenance of the elastics.

Based on the information and instructions provided, the patient may practice putting the elastics on using a computer simulation to ensure that they understand the instructions. In one preferred embodiment, a custom length elastic can be created using a three-dimensional printer (“3D printer”) and sent to the patient. In another preferred embodiment, the central processor 5 selects standard or off-the-shelf elastics that provide the closest length and tension properties to the ideal elastic and directs the patient regarding the positioning and timing for use of the elastics.

The preferred method improves the prior art orthodontic systems and methods by providing a level of precision in elastic selection (length and tension), among other improvements. Clinicians often install the elastics and find that they are not the correct length for the patient and then must remove the elastics and try a different length elastic. The patient can perform this procedure from home, rather than necessitate an office visit and the associated waste of time travelling to the appointment, potentially at a location significantly distanced from the patient's location. The central processor 5 may also send multiple standard sized elastics for the patient to use so that failure of one or more of the elastics based on improper sizing or tension does not result in failure to utilize the elastics, as an incorrect or failed elastic may be removed and replaces by a relatively close in size and tension elastic until the appropriate elastic is applied to the patient's braces.

The preferred invention also facilitates an automated check-out step 60 for the patient that is preferably performed after a Treatment Execution step 50 in either the clinical location, at the patient's home or at nearly any location where the patient is positioned. In the preferred check-out, images of the patient's teeth, arches and/or face are taken, preferably by the patient. The captured images are uploaded to the patient record at the central processor 5. Evaluation of the images is conducted by the software or software modules of the central processor 5, potentially including image processing routines, “AI” or machine learning. The evaluation preferably compares the treatment prescription, as detailed in the patient record, to the imaging to determine at least one of: (1) identifying which arch wires 136 are in the patient's mouth, (2) whether there is a stop installed on the archwire 136 and whether the stop 140 is appropriately positioned, (3) whether all brackets 138 prescribed are still attached, (4) whether any brackets 138 are damaged or appear to be loose, (5) whether any brackets 138 are out of position relative to the 3D model detailing the optimal bracket placement which is stored in the patient's treatment records, (6) whether each bracket 138 is properly engaged as is described in further detail herein (See at least ¶32), (7) whether the prescribed elastic is being used and if it is mounted appropriately on the bracket posts prescribed and (8) any additional requirements or requests that are included in the patient's record or in the standard operating procedure included in the central processor 5. To determine if the elastic is the correct one or appropriate for the specific patient and the treatment plan, the software of the central processor 5 preferably reviews any visible marking (color or model markings) on the elastic or measures the thickness of the elastic, which also can vary based on how much tension the elastic is under. Measuring the distance assists in determining how much tension the elastic is under and preferably assists in determining a thickness and length for a potential suggestion of an elastic. The elastics may further be evaluated by the central processor by directing the patient to collect images of the elastics unloaded or outside of the patient's mouth so that the central processor 5 can determine or estimate the size and tension properties of the elastics and direct the patient to apply specifically identified elastics based on the collected images.

The preferred system and method also preferably evaluate and determines whether there is evidence of gum swelling or recession for the patient during treatment. The system preferably measures the overall length of the patient's arch and specific distances between teeth and/or center-points of the brackets and once the evaluation is complete the results are recorded in the patient record and communicated to one or more of the patients and clinicians who performed the treatment. The results are communicated to the patient or clinician on at least one of: a computer, tablet or cell phone, accessible electronic file or audibly. The results of the evaluation are preferably recorded in the patient record at the central processor 5 and communicated as “satisfactory” (complete) or “unsatisfactory” (required further action). If further action is required, the system may communicate specific actions required to complete the treatment satisfactorily. This routine may be repeated until such time as the evaluation is considered complete.

The preferred check-out routine is a significant improvement over current orthodontics techniques performed in known and well-established orthodontics techniques. The preferred system and method are faster than a manual and visual check, more accurate—with current treatments careless errors occur when checking the patient chart vs. what was done, virtually impossible for the human eye to differentiate the wire lengths, sizes, shapes and fit, can be done at any time, including during non-clinical hours, can be done remotely thus far more convenient for the patient but can also be done in a clinical office and additional improvements. Many conventional orthodontic offices skip the check-out by the clinician and do not check the work of less skilled dental assistants who typically do the adjustments. This less than ideal procedure leads to errors that can result in damage to the patient's teeth or which may impede the progress of the overall treatment. Reasons that certain clinicians would skip this step include laziness, difficulty in scheduling multiple patients in the office at the same time with the clinician overseeing a staff of assistants and causing patients to wait for the clinician to be available and check the assistant's work. Checking that the wires 136 are engaged and that the distal ends are not protruding such that they don't poke the patient in the checks requires the clinician to put their hands in the patient's mouth to physically feel the wires 136.

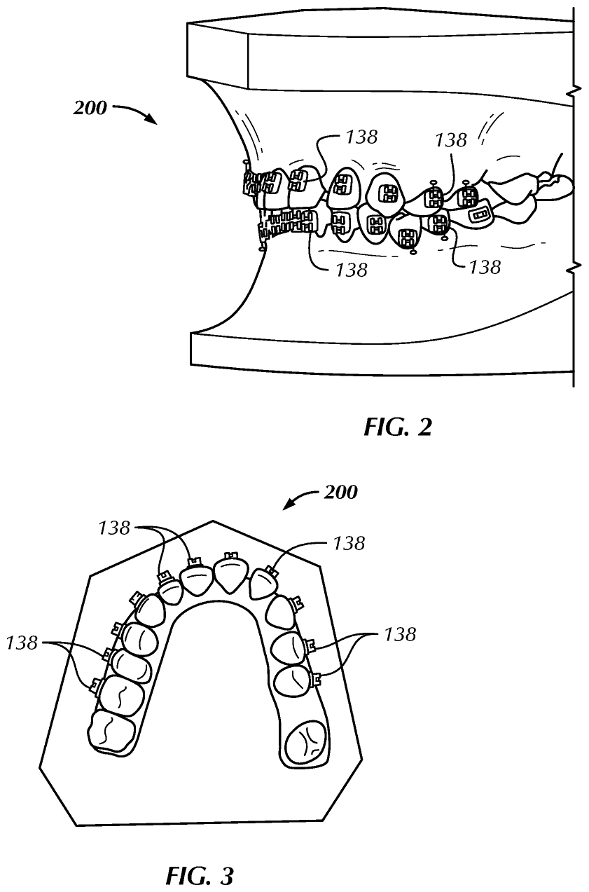

Referring to FIGS. 1 and 2, the preferred system and method uses the software modules of the central processor 5 to create a 3D image or model 200 of the patient's arches and allows the clinician to develop a treatment plan and place the brackets 138, either virtually or physically, in their ideal location on the arches or the 3D image or model 200 to effectively move the teeth during the treatment. The software of the central processor 5 is utilized to create bonding transfer trays but can also be used as a visual guide for on-site clinicians to use as a reference for placing and bonding the brackets 138 by hand, particularly by providing access to the 3D image or model 200 to the on-site clinician to assist in the placement of the brackets 138 and wires 136 (See also at least ¶17).

The preferred hardware and form factors of the image capture routine and upload step 10 are described for use in developing the treatment plan during the check-in & evaluation step 20 (See also at least ¶27).

The preferred system and method determine what wires 136 are going in the patients mouth and materials and thickness thereof based on the image capture and upload of the images that may be performed by the patient or an assistant during the upload step 10. The wires 136 themselves may be made of nitinol and/or stainless steel in gauges from twelve to twenty thousandths (0.012-0.020) in either round or rectangle shape. In the case of the nitinol wires 136, the nitinol wires 136 are preferably pre-formed in the shape of the patient's arch. The system and method are utilized to identify wires 136 by various features, such as diameter, shape, material, etc., via imaging to determine their material and/or gauge, wherein such features may include at least one or more of: a coating on a section of the wire 136 in a distinct color which dissolves or fades relatively quickly so that the coating or color is not visible (unsightly to patient) after a limited amount of time following installation. Such coating is preferably benign to human ingestion and may be constructed of any biocompatible material that is able to perform the preferred functions of the coating or color and withstand the normal operating conditions of the coating or color (See also at least ¶31).

The identification feature on the wire 136 that indicates where the wire 136 should be placed relative to the brackets 138 may also be comprised of a non-visible coating on the wire 136 in the non-visible light spectrum covering all or a portion of the wire 136. Such non-visible coding may include a bar-code like series of lines to denote the type of wire 136. A permanent color coating situated on only a small portion of the wire 136 so that it is not easily visible may also be utilized to align the wire with the brackets for proper placement.

The software of the preferred system that is preferably stored at least partially in the central processor 5 may be programmed with the ability to measure the diameter and/or shape of the wire, features for identifying material based on color, sheen or texture and other features for performing the functions of the preferred system and method.

The preferred system utilizes monitoring to determine whether the wires are properly engaged into the brackets after installation. (See also at least ¶32). The treatment typically involves one or more of the bracket types listed below, each of which engages the wire 136 in a distinctive manner based on the brackets 138, wires 136 or other factures. The preferred invention involves using high resolution imaging or other imaging and evaluation by the software of the central processor 5 to determine at least one of: (1) in the case of the self-ligating brackets 138 that a cross section of the archwire 136 engaged into each bracket 138 is not visible which can only happen if the “gate” of the bracket 138 is fully closed thus obscuring that section from view, (2) in the case of a traditional bracket 138 that the elastic ligature used to engage the bracket 138 is engaged securely, wherein normal engagement would be from four points around the bracket 138, but can be a minimum of two points of engagement and still be adequate (Drawing needed), and/or (3) in the case of molar brackets 138 where the wire 136 is fully engaged into molar bracket 138 and that it is not extending distally from the tube more than a predetermined distance, wherein the limiting the extension of the wire 136 at the molar tubes is to prevent the wire 136 from poking the patient's check and gums.

Absent the preferred invention, in order for a clinician to make this evaluation of each of the bracket 138 varieties and each of the connections, in-person, the clinician normally would have to manually look at and “test” each bracket 138 using either their fingers or a handheld dental tool to ensure that the wire 136 and bracket a138 re properly engaged. This review, check and test of each of the connections is time consuming and interrupts the flow of patient treatments in the office since normally there are multiple patients receiving treatment in the clinician's office at the same time. This review and check can also lead to; (a) some clinicians skipping this important step and/or (b) errors due to the small sizes of the brackets 138, the positioning of the brackets 138 in the mouth and the large number of individual tooth checks necessary per patient and per day by the clinician. The potential errors introduced during the review and check is particularly a concern when many patients are being treated in an office on a daily basis.

The preferred system also preferably evaluates the health of the patient's gums during the orthodontic treatment, which can be difficult for a patient because of obstructed care and maintenance due to the orthodontic hardware in the patient's mouth and other factors. (See also at least ¶34). The preferred system measures the visible portion of each tooth in relation to the patient's gums and compares the multiple captured surface areas to the images and measurements from prior appointments. If the gum is swollen, the gum will typically obscure more of the tooth and less tooth surface will be visible. Conversely if the gums are recessed, the opposite consequence occurs. The software of the central processor 5 also preferably compares the color and hue of the gums to at least one of: (1) prior appointment images for the patient or (2) a database of “normal hue” for a healthy gum (compiled from a wider patient population). Information from this evaluation can be displayed in the patient record and communicated to the patient, clinician or other individuals who are authorized for such information. This will enable one of the clinician or software modules of the central processor 5 to visually show the patient the specific area of the gums which are of concern and recommend proactive measures to improve the condition identified.

The preferred system also tracks and analyzes arch length and length of the wires 136 as they change dynamically throughout treatment. (See also at least ¶36). Tracking and monitoring the patient's gums can be particularly important during orthodontic treatment, as patient's may have difficulty appropriately caring for their gums and the areas between the orthodontic hardware and the gums during treatment due to the orthodontic hardware mounted in their mouth and general soreness and discomfort resulting from treatment. The effectiveness of various treatment methods and systems for patient gum maintenance may also be monitored and evaluated by the upload step check-in & evaluation step 20, decision matrix step 30, evaluation step 40, treatment execution step 50 and check-out step 60 of the preferred present invention.

The preferred system determines whether a new, incremental treatment recommendation is warranted during the treatment based on the various images that are collected and saved during treatment. For example, the patient's arches may have moved as much as they can from a particular arch wire 136 or that an elastic configuration has moved the teeth sufficiently. The clinician, potentially via the software of the central processor 5, can also project the timing when such movements of the patient's teeth will necessitate a change in treatment and recommend the timing for the next check-in or appointment. Among other things the clinician may determine whether; a) new wires 136 or a change in wire type shape or thickness are necessary, b) use of or a change of inter-arch or intra-arch is indicated and other related treatment modifications. The clinician can also project the timing when such movements will necessitate a change in treatment and recommend the timing for the next check-in or appointment.

The preferred system is able to display various information, images and related materials associated with the treatment to the patient and clinicians. Treatment recommendations, such as size, shape, material and customized length of the archwires 136 (length can be displayed in numeric form and/or with a visual image of the customized wire length), elastics type (length and tension), elastics configuration (what teeth are engaged and which posts on the brackets, particular hygiene concerns pinpointing specific regions of the mouth), recommendations for oral hygiene such as softer or more vigorous brushing, flossing, oral rinses, type of brush utilized and related features. The display can include instructions using at least one of text, still images, videos or animation. The preferred embodiment can also include an interactive display that allows the patient to practice the suggested procedure developed by the clinician on-screen before performing the suggested procedure in their own mouth or with assistance from a third-party helper. The display may employ various instruction methods and features, such as text, video, still images, animations, models and related instruction displays. When displaying a feature, such as the customized wire length, the patient can hold up the actual wire 136 they are going to put into the mouth against the display as a point of comparison so that the system is able to confirm that the two sizes match. Such display of the archwire 136 may include a marking as to where a stop 140 should be placed and locations where the archwire 136 should be engaged with the brackets 138. The interactive display may also allow the patient to practice the procedure virtually on-screen before conducting the procedure in their own mouth. The stop 140 may be placed on and engaged with the wire 136 at a predetermined location to prevent the wire 136 from moving in a specific direction relative to the brackets 138 or to generally limit movement of the wire 136 relative to the brackets 138 when mounted, particularly if multiple stops 140 are applied to the wire 136.

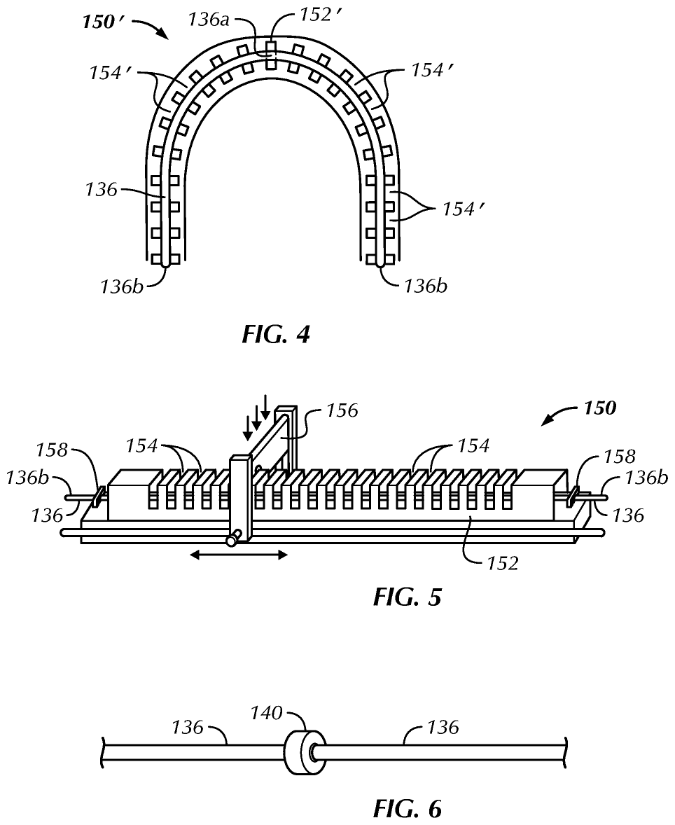

Referring to FIGS. 4 and 5, details regarding the preferred method for the patient to obtain or adjust the archwires 136 to their custom prescribed length may be determined by the preferred system. The system may include a jig 150, 150′ where patients can insert the wire 136 with a centerline 136a positioned in the center of the jig 150, 150′ and cut the distal end(s) 136b along certain pre-determined lengths according to the instructions provided by the system. The wires 136 may also have a series of markings on the distal ends 136b of the wire 136 and the patient can be instructed by the preferred system which length to clip the archwire 136 using the jig 150, 150′ so that the archwire 136 is positionable and engageable with the patient's brackets 138. The preferred system includes a preferred jig 150 and an alternative preferred jig 150′, wherein the preferred jig 150 is adapted for sizing a relatively straight wire 136 and the alternative preferred jig 150′ is configured for sizing an arcuate wire 136 that is generally adapted to the shape of a typical patient's arches. Similar features of the preferred and alternative preferred jigs 150, 150′ are identified with the same reference number with the features of the alternative preferred jig 150′ distinguished by a prime symbol (“′”).

The patient can customize the length of the archwire 136 themselves based on the length recommendation of the remote clinician and based on messages and instructions from the central processor 5. The patient may be provided with a standard length archwire 136 or a kit of standard length archwires 136 of different gauges shapes and material, as well as with the jig 150, 150′ and then will cut the archwires 136 themselves to the prescribed length using the jig 150, 150′. The jig 150, 150′ preferably has a center point 152, 152′ wherein the centerline 136a of the wire 136 is centered in the jig 150, 150′ and secured so that the wire 136 does not move or shift during the cutting process. The jig 150, 150′ preferably has a series of notches 154, 154′ extended inwardly from the outermost portion of the jig 150, 150′ (which would correlate to the distal ends 136b of the archwire 136) to the midpoint or centerpoint 152, 152′ of the jig 150, 150′. The instructions to the patient from the central processor 5 preferably will indicate which notch 154, 154′ should be used on both sides of the jig 150, 150′ to trim the wire 136 at the distal ends 136b. A cutting device 156 can be a flat guillotine type apparatus which can be incorporated into the jig 150, 150′ so that it slides from position to position or the cutting device 156 can be something as simple as a small wire clipper device, crimper, scissors or other device that is able to sever the wire 136. The jig 150, 150′ preferably includes a securing mechanism 158 that engages the wire 136 so that the wire 136 does not shift while being cut by the cutting device 156. The securing mechanism 158 may be comprised of a clamp used to secure the wire 136 in one or more positions, a fastener, adhesive or other securing method or system that secures the wire 136 in place relative to the jig 150, 150′ during the cutting process. The jig 150, 150′ is in the shape of the archwire 136 to minimize the deformation of the archwire 136 while in the jig 150, 150′, but the jig 150, 150′ can also be in other shapes such as being curved in multiple directions or adjustable to the shape of the wire 136. The cutting device 156 may be mounted on a track on the jig 150, 150′ that is movable relative to the notches 154, 154′ such that the user is able to move the cutting device 156 to the desired notches 154, 154′ to cut the wire 136. The cutting device 156 may be securable or lockable to the jig 150, 150′ at positions in alignment with the notches 154, 154′.

In the preferred embodiment the archwire 136 will be customized to the patient's arches. Archwires 136 are manufactured in standard lengths and then need to be trimmed. The patient inserts a standard length archwire 136 into the jig 150, 150′ and trims the wire 136 to the desired length reducing the length of the wire 136 in roughly even increment from the distal ends 136b of the wire 136. Alternatively, the archwire 136 may have a series of distinctive markings on them which would serve as guides or reference points for the patient to trim the wires 136 by hand.

Referring to FIGS. 4-9, the preferred system preferably recommends or directs how the stops 140 are rigidly pre-mounted to the wire 136 in a custom prescribed location, and/or in an alternative embodiment a crimp stop 140 can be a donut shaped stop 140 pre-mounted to the wire 136 under some tension which can, with manual force, slide on the wire 136 to the optimal location and then be crimped permanently to prevent the archwire 136 from shifting relative to the brackets 138 during use. The stop 140 is not limited to the described configurations or the configuration illustrated in FIG. 6 and may be constructed having nearly any shape or material, such as cylindrical, square, cubic, disc-shape, donut-shaped, steel, polymeric or other shapes and materials, respectively. The preferred stop 140 has a general donut-shape that is slidable on the wire 136 and may be crimped, snapped or clamped onto the wire 136 in a desired position. The stop 140 may have distinctive colors, shapes or printed markings that differentiate the stops 140 used for specific types of wires 136, but the stop 140 may also be universally designed and configured. The stops 140 may also be adapted for use with the jigs 150, 150′ by directing placement of the stops 140 based on the notches 154, 154′ for appropriate placement of the stops 140.

Referring to FIGS. 1A-10C, the preferred invention is also directed to a method for evaluating a patient's orthodontic treatment following application of a plurality of brackets 138 to the patient's teeth and mounting a wire 136 to the plurality of brackets 138 wherein the plurality of brackets 138 and the wire 136 are applied to the patient's teeth based on a first treatment plan. The patient or a clinician takes images of the patient's teeth with the brackets 138 and wire 136 attached thereto, the images are collected by the image capture device 118 and the collected images are received at the central processor 5. The image capture device 118 may be comprised of a camera, a smartphone, a tablet or another device that is able to capture images of the patient's teeth and arches, at least temporarily store the images and transmit the images to the central processor 5. In preferred embodiments, the image capture device 118 may include a smartphone that is tethered to a camera via a wired or wireless protocol. The images preferably include left, center and right buccal images to capture at least one images of each of the brackets 138 and the full length of the wire 136 but are not so limited and may be comprised of less or more images that the central processor 5 is able to utilize to check the brackets 138 and wire 136 or other dental hygiene or anatomic features of the patient. For example, the images may be comprised of a single panoramic image that is able to capture images of each of the brackets 138 and the wire 136 mounted to the patient's teeth. The preferred image capture device 118 may include an image screen (FIG. 9) that the patient or clinician may utilize during the capture of the images to observe the images being captured on the image screen. The image screen may also include customized prompts from the central processor 5 directing the patient or the clinician collecting the images regarding the number of images to collect and the orientation of the images. The customized prompts sent from the central processor 5 to the image screen may include a straight-on shot with the patient looking at the image capture device 118 or using a panoramic sweep motion with the image capture device 118 traversing a generally U-shape around the teeth. The customized prompts are not limited to the straight-on shot and the panoramic sweep and may be comprised of nearly any image angle and width or combination that is able to provide appropriate images to the central processor 5 for making recommendations for the patient's treatment based on movement of the patient's teeth and/or positioning of the teeth.

The preferred method also includes capturing the images with the assistance of the image capture device 118 using or with an image capture guide 110, which may include the cheek retractor 120. The image capture guide 110 is configured to orient the image capture device 118 relative to the patient's teeth and consistently space the image capture device 118 relative to the patient's teeth. Consistent spacing of the image capture device 118 relative to the patient's teeth and generally consistent repeating of the images from successive image capture appointments or sessions assists the central processor 5 in evaluating the images between appointments to accurately determine tooth movement between appointments and as a result of the treatment plans. Consistent orientation and spacing of the image capture device 118 relative to the patient's teeth provides generally consistent images so that the central processor 5 or clinician is able to accurately visualize the progress of the treatment plan and development of future or updated treatment plans, as well as anticipated timing for the treatment. In a preferred embodiment, the image capture guide 110 is comprised of a chin rest 112 and an actuating arm 116, and the image capture device 118 is connected to the actuating arm 116. The image capture guide 110 is not limited to including the chin rest 112 or the actuating arm 116 and may be comprised of nearly any structure or operating instruction that facilitates consistent spacing of the image capture device 118 from the teeth and repeatability of the images of the patient's teeth that are captured. For example, the image capture guide 110 may be comprised of prompts and sensors provided by and in communication with the central processor 5 that directs the patient or clinician to move the image capture device 118 relative to the patient's teeth and directs the patient or clinician when to capture the images. The image capture guide 110 may also be comprised of nearly any hardware or physical guide that consistently positions and orients the image capture device 118 relative to the patient's teeth that results in relatively consistent images being transmitted to the central processor 5 when images are collected. The image capture guide 110 may also include the cheek retractor 120 that is configured to create distance between the soft tissue and the patient's teeth to produce relatively clear and consistent images of the patient's teeth when the images are collected by moving soft tissue, such as the patient's cheeks away from the teeth and gums.

The preferred method also includes comparing the collected images of the patient's teeth with the plurality of brackets 138 mounted thereon and the wire 136 to the first treatment plan, which includes previous images of the patient's teeth stored in the central processor 5. As described above, the comparison preferably involves comparing the brackets 138 and wire 136 to the expected brackets 138 and wire 136 of the treatment plan or the previously collected images to identify error conditions or irregularities between the actual collected images and the previous images or the expectations of the treatment plan. The comparison may be aided by a coating, printed lines or an indentation that identifies a wire diameter and/or length on the wire 136. The central processor 5 may determine whether the wire 136 is an identified wire of the first treatment plan in this comparing step based on the coating, printed lines or indentation, by comparing the coating, printed lines or indentation to a lookup table in the central processor 5 that identifies the wire 136. The coating on the wire 136 may be comprised of a color that will dissolve or fade relatively quickly after exposure to the environment of the patient's mouth, a coating in a non-visible light spectrum or a permanent color on a portion of the wire 136 that assists with identification of the wire 136 from the images.

The preferred method further includes transmitting a message from the central processor to the patient, preferably to the image capture device 118, regarding an error condition or a confirmation condition. The error condition may be comprised of any errors or deviations from the treatment plan or expected images of the patient's teeth or arches. As non-limiting examples, the error condition may be comprised of a first number of brackets 138 of the first treatment plan not being equal to an identified number of brackets 138 in the images of the patient's teeth, a bracket 138 in the images being identified as open in the images, an end of the wire 136 extending beyond a last molar bracket 138 of the plurality of brackets 138 in the images, a wire size of the wire 136 in the images deviating from a plan wire size of the first treatment plan or a position of a gum line of the patient in the images deviating from a plan position of the first treatment plan. These example error conditions are not limiting and may be nearly any other deviation from the first treatment plan identified in the images collected of the patient's teeth. The message from the central processor may include an indication of a specific area of the patient's gums that are of concern and recommended proactive measures to improve the specific area of concern, such as prompts to the patient to pay particular attention to brushing in the specific area of concern. The message may also include suggestions for making a follow-up appointment with a clinician, instructions regarding how to correct a deviation, such as changing a wire 136, closing a bracket 138, cutting a different length wire 136 using the measuring tool 150, 150′, incorrect positioning of the elastics or nearly any other deviation from the treatment plan that the central processor 5 is able to identify based in the collected images. An updated treatment plan is preferably developed based on the images of the patient's teeth. The updated treatment plan preferably includes a recommended date for a patient to attend an appointment in a clinical setting for an adjustment or the recommended date may be for a patient to collect and send additional images to the central processor 5 for evaluation of the progress of the treatment. The recommended date may be comprised of multiple dates and times for selection by the patient, a date range for capturing images with the image capture device 118 or other recommendations for the patient to further the treatment. If new images are requested with the updated treatment plan, the new images are preferably taken from the same or similar angles, in the same sequence and the same or similar distances from the patient's teeth as the previous images for ease of comparison by the central processor 5. The message also preferably includes instructions regarding where and when subsequent treatment of the teeth is planned in the updated treatment plan, whether the treatment will be at a clinical setting, at the patient's discretion or when additional images are scheduled for collection by the patient or the clinician.

The preferred method may include sending a custom length wire 136 and elastics to the patient with instructions regarding which of the elastics to apply to the plurality of brackets 138, which posts may be used to attach the elastics, how to apply the elastics and how to install the custom length wire 136. The method is not limited to sending custom length wires 136 and the patient may receive a relatively large wire that with instructions to cut to a specific length using the cutting and measuring tool 150, 150′, where and how to apply the stops 140, how to install the wire 136 and which images to take with the image capture device 118 so that the central processor 5 is able to check the images against the treatment plan, as is described herein.

A preferred embodiment may also be directed to a method for automated check-out of a patient performed after execution of an orthodontic treatment on the patient including application of brackets 138 and an archwire 136 to the patient's teeth. The method includes the step of receiving images of the patient's arches at the central processor 5 and evaluating the images of the patient's arches at the central processor 5. The preferred images include left, center and right buccal images that visualize or include images of each of the brackets 138 installed on the patient's teeth and the full length of the archwire 136. The images are preferably captured with the image capture device 118. The evaluation of the images involved comparing the images of the patient's arches to a treatment plan stored in the central processor 5. The treatment plan includes a predetermined number of brackets, a predetermined bracket position, and a predetermined archwire 136. The comparison also includes identifying a number of brackets 138 in the images of the patient's arches, a bracket position in the images of the patient's arches, and an archwire 136 in the images of the patient's arches and comparing the predetermined number of brackets 138 to the number of brackets 138, the predetermined bracket position to the bracket position and the predetermined archwire 136 to the archwire 136. The method also includes transmitting a check-out message from the central processor 5 indicating whether the predetermined number of brackets is equal to the number of brackets, the predetermined bracket position complies with the bracket position and the predetermined archwire 136 complies with the archwire 136. The check-out message may be comprised only of a verification or confirmation that the comparison is acceptable without any additional detail or may include additional detail regarding the results of the comparison. The check-out message may also be comprised of an error message if the comparison identifies a failure in the comparison, an identification of other errors, such as gum disease identification, tooth decay or other errors identified by the comparison with the central processor 5.

In addition to the left, center and right buccal images taken with the image capture device 118, the images may include additional images, such as mandibular occlusal and maxillary occlusal images or other images that are preferred or desired by the clinician or orthodontist. The images are preferably captured with the assistance of the image capture guide 110 that is configured to orient the image capture device 118 relative to the patient's arches and consistently space the image capture device 118 relative to the patient's arches.

The central processor 5 preferably determines when new archwires 138 are recommended and the updated treatment plan for the patient. The central processor also determines positioning of the stop 140 on the archwire 138 and whether the stop 140 is installed on the archwire 138 in the appropriate location or predetermined location during the evaluation step. The central processor 5 may be comprised of software that makes determinations based on the images and the comparison or a clinician that manually conducts the comparison and develops the updated treatment plan and recommendations for the patient in the check-out message. The step of evaluating the images preferably includes reviewing positioning of the patient's gums relative to the patient's teeth and estimating gum swelling, particularly with respect to previous images taken of the patient's gums and teeth and stored in the central processor 5. The check-out message preferably includes instructions for treatment of the patient's gums designed to improve the patient's gum health. The check-out message of the preferred embodiment also may include a recommended date for the patient to attend a subsequent appointment or to capture additional images of the patient's arches for additional comparisons, evaluations and to check for error conditions in the orthodontic hardware, the assembly, the patient's dental health or any additional comparisons or error conditions that may be detected by the central processor 5.

It will be appreciated by those skilled in the art that changes could be made to the embodiment described above without departing from the broad inventive concept thereof. It is understood, therefore, that this invention is not limited to the particular embodiment disclosed, but it is intended to cover modifications within the spirit and scope of the present invention as defined by the present disclosure.

1-23. (canceled)

24. A method for evaluating a patient's orthodontic treatment following application of a plurality of brackets to the patient's teeth and mounting a wire to the plurality of brackets wherein the plurality of brackets and the wire are applied to the patient's teeth based on a first treatment plan, the method comprising the steps of:
receiving images of the patient's teeth at a central processor from an image capture device, the images being captured with the assistance of an image capture guide having an arm that is configured to orient the image capture device relative to the patient's teeth outside of the patient's mouth and consistently space the image capture device relative to the patient's teeth;
comparing the images of the patient's teeth with the plurality of brackets mounted thereon and the wire to the first treatment plan and previous images of the patient's teeth stored in the central processor; and
transmitting a message from the central processor to the patient regarding an error condition or a confirmation condition, an updated treatment plan being developed based on the images of the patient's teeth.

25. The method of claim 24, wherein the error condition is comprised of a first number of brackets of the first treatment plan is not equal to an identified number of brackets of the images of the patient's teeth, a bracket in the images is identified as being open, an end of the wire extends beyond a last molar bracket of the plurality of brackets in the images, a wire size of the wire in the images deviates from a plan wire size of the first treatment plan, or a position of a gum line of the patient in the images deviates from a plan position of the first treatment plan.

26. The method of claim 24, wherein the image capture device is comprised of a camera, a smartphone or a tablet.

27. The method of claim 24, wherein the image capture guide is comprised of a chin rest and the arm, the arm comprised of an actuating arm, the actuating arm including a horizontal arm connected to the chin rest and a pivoting arm, the image capture device connected to the actuating arm.

28. The method of claim 24, wherein the image capture guide includes a cheek retractor configured to create distance between soft tissue and the patient's teeth to produce relatively clear and consistent images of the patient's teeth.

29. The method of claim 24, wherein the images include left, center, and right buccal images.

30. A method for evaluating a patient's orthodontic treatment following application of a plurality of brackets to the patient's teeth and mounting a wire to the plurality of brackets wherein the plurality of brackets and the wire are applied to the patient's teeth based on a first treatment plan, the method comprising the steps of:
receiving images of the patient's teeth at a central processor from an image capture device, the images being captured with the assistance of an image capture guide that is configured to orient the image capture device relative to the patient's teeth and consistently space the image capture device relative to the patient's teeth;
comparing the images of the patient's teeth with the plurality of brackets mounted thereon and the wire to the first treatment plan and previous images of the patient's teeth stored in the central processor;
transmitting a message from the central processor to the patient regarding an error condition or a confirmation condition, an updated treatment plan being developed based on the images of the patient's teeth; and
providing elastics to the patient with instructions regarding which of the elastics to apply to the plurality of brackets.

31. The method of claim 24, wherein the image capture device includes an image screen, during the capture of the images the patient observes the images being captured on the image screen, the image screen also including customized prompts from the central processor directing the patient regarding a number of images and orientation of images.

32. The method of claim 31, wherein the customized prompts include a straight-on shot with the patient looking at the image capture device and using a panoramic sweep motion with the image capture device traversing a generally U-shape around the teeth.

33. The method of claim 24, wherein the updated treatment plan includes a recommended date for the patient to attend an appointment at a clinical setting for an adjustment.

34. The method of claim 24, wherein the image capture device is comprised of a camera tethered to a smart phone.

35. The method of claim 24, wherein the updated treatment plan includes a suggested date for submission of new images of the patient's teeth, the new images being taken in the same sequence as the images of the patient's teeth.

36. The method of claim 24, wherein the message includes an indication of a specific area of the patient's gums that are of concern and recommended proactive measures to improve the specific area of concern.

37. The method of claim 24, wherein the message includes instructions regarding where and when subsequent treatment of the teeth is planned in the updated treatment plan.

38. The method of claim 24, wherein the wire includes a coating, printed lines or an indentation that identifies a wire diameter, the central processor determines whether the wire is an identified wire of the first treatment plan in the comparing step based on the coating, printed lines or the indentation.

39. The method of claim 38, wherein the coating is comprised of a color that will dissolve or fade relatively quickly, a coating in a non-visible light spectrum or a permanent color on a portion of the wire.

40. A method for automated check-out of a patient performed after execution of an orthodontic treatment on the patient including application of brackets and an archwire to the patient's teeth, the method comprising the steps of:
receiving images of the patient's arches at a central processor, the images including left, center and right buccal images, the images being captured with an image capture device;
evaluating the images of the patient's arches at the central processor, the evaluation involving comparing the images of the patient's arches to a treatment plan stored in the central processor, the treatment plan including a predetermined number of brackets, a predetermined bracket position and a predetermined archwire, the comparison including identifying a number of brackets in the images of the patient's arches, a bracket position in the images of the patient's arches and an archwire in the images of the patient's arches and comparing the predetermined number of brackets to the number of brackets, the predetermined bracket position to the bracket position and the predetermined archwire to the archwire; and
transmitting a check-out message from the central processor indicating whether the predetermined number of brackets is equal to the number of brackets, the predetermined bracket position complies with the bracket position and the predetermined archwire complies with the archwire.

41. The method of claim 40, wherein the evaluating the images also includes reviewing positioning of the patient's gums relative to the patient's teeth and estimating gum swelling, the check-out message includes instructions for treatment of the patient's gums.

42. The method of claim 40, wherein the images include mandibular occlusal and maxillary occlusal images.

43. The method of claim 40, wherein the images are captured with the assistance of an image capture guide that is configured to orient the image capture device relative to the patient's arches and consistently space the image capture device relative to the patient's arches.

44. The method of claim 40, wherein the check-out message includes a recommended date for the patient to attend a subsequent appointment or to capture additional images of the patient's arches.

45. The method of claim 40, wherein the central processor determines when new archwires are recommended and an updated treatment plan for the patient, the central processor also determines positioning of a stop on the archwire.

46. The method of claim 45, wherein the central processor determines whether the stop is installed on the archwire during the evaluating step.

47. The method of claim 27, wherein the actuating arm is comprised of a telescopic arm.

48. The method of claim 40, further comprising:
providing a custom length wire to the patient with instructions regarding which posts to be used to attach the elastics, how to apply the elastics and how to install the custom length wire.
